(19) United States
(12) Patent Application Publication
(10) Pub. No.: US 2006/0235077 A1
(43) Pub. Date: Oct. 19, 2006

(54) Diverticular remodling technique

(76) Inventor: Syde A. Taheri, Williamsville, NY (US)

Correspondence Address: WALTER W. DUFT;LAW OFFICES OF WALTER W. DUFT, 8616 MAIN ST, SUITE 2, WILLIAMSVILLE, NY, 14221, US

(21) Appl. No.: 11/181,671

(22) Filed: Jul. 14, 2005

Related U.S. Application Data
(60) Provisional application No. 60/672,130, filed on Apr. 13, 2005.
(60) Provisional application No. 60/680,182, filed on May 12, 2005.

(51) Int. Cl. A61K 31/225; A61M 31/00; A61K 31/765

(52) U.S. Cl. 514/547; 604/500; 424/78.37

(57) ABSTRACT

A diverticular remodeling technique comprises the introduction of a remodeling agent into the diverticulum to promote permanent closure of the diverticular sac. In disclosed exemplary embodiments, the diverticulum is accessed non-invasively and optionally cleaned to remove unwanted matter therefrom. The remodeling agent is introduced into the diverticular interior and the neck of diverticulum is closed (as necessary) to prevent egress of the remodeling agent. The remodeling agent can be any of a variety of materials, including small intestine submucosa (SIS) collagen or other absorbable fibrosis materials in gel, liquid or solid form. An endoscopic device may be used that includes a multi-lumen flexible catheter having a distal end adapted to be steered through a patient and a proximal end that remains outside the patient's body.

### CROSS-REFERENCE TO RELATED APPLICATIONS

This application claims benefit of the filing date of U.S. Provisional Application No. 60/672,139, filed Apr. 13, 2005, and U.S. Provisional Application No. 60/680,182, filed May 12, 2005.

### STATEMENT REGARDING FEDERALLY SPONSORED RESEARCH OR DEVELOPMENT

Not Applicable

### BACKGROUND OF THE INVENTION

1. Field of the Invention

This invention relates to the treatment of diverticular ailments, such as diverticulosis and diverticulitis of the colon.

2. Description of the Prior Art

By way of background, as a person ages his or her colon can develop flask-like structures that extend from the lumen through the surrounding muscle layers. These structures, which typically range in size from 5 to 10 mm up to 2 cm, are known as diverticula and their presence in the colon is referred to as diverticulosis. The wall of each diverticulum is in continuity with surface mucosa and displays an epithelium and sub-mucosa. The base of a diverticulum is formed by serosal connective tissue. Diverticulosis is an age-dependent disease that is relatively uncommon in persons under age 40. It is also unusual to find this condition in Asia, Africa and underdeveloped countries. However, in Western societies, approximately 10% of all persons are afflicted. Between the ages of 40-60, the occurrence rate is approximately 5%, but this rate increases to an astonishing 65% by the time a person reaches age 65 and higher. In 1980, diverticulosis affected 30 million Americans and resulted in 200,000 hospital visits. This number continues to increase.

A typical case of diverticulosis may involve anywhere from one to a dozen diverticula. The presence of these structures in the colon is undesirable because the condition can lead to complications such as bleeding and an inflammatory condition known as diverticulitis. Of all persons afflicted with diverticulosis, approximately 5-15% will develop problems with bleeding and 5 to 20% will experience episodes of diverticulitis. The term diverticulitis was used as early as 1899. The condition develops when bowel contents become trapped in one or more of the divercula, causing infection, inflammation and acute pain. In the early part of the twentieth century, diverticulitis was treated by surgical intervention. Today, the most common course of treatment is antibiotics, with surgical intervention occasionally being necessary to remove impacted material from the affected diverticula. Increased dietary fiber intake is believed to help prevent diverticulitis in persons who have diverticulosis, and also to help control the formation of new diverticula.

In addition to the colon, diverticula may also be found in other areas of the body, including the small intestine, and the esophagus. As in the colon, these diverticula can cause problems, such as diverticulosis/diverticulitis in the small intestine. In the esophagus, divertular rupture can result in aspiration of esophageal material into the trachea.

It is to improvements in the treatment of conditions associated with the presence of diverticular structures in the body that the present invention is directed. In particular, what is needed is a new technique whereby diverticular structures can be effectively mitigated without surgery using standard endoscopy equipment.

### SUMMARY OF THE INVENTION

An advance in the art of diverticular treatment is provided by a diverticular remodeling technique that comprises the introduction of a remodeling agent into a diverticulum to promote permanent closure of the diverticular sac. In exemplary embodiments of the invention disclosed herein, the diverticulum is accessed non-invasively and optionally cleaned to remove unwanted matter therefrom. The remodeling agent is introduced into the diverticular interior and the neck of diverticulum is closed (as necessary) to prevent egress of the remodeling agent. The remodeling agent can be any of a variety of materials, including small intestine submucosa (SIS) collagen or other absorbable fibrosis promoting material in gel, liquid, or solid form.

For colonic diverticular remodeling, an endoscopic device may be used that includes a flexible catheter having a distal end adapted to be steered through a patient and a proximal end that remains outside the patient's body. The flexible catheter may include a plurality of lumina for performing a variety of tasks, such as a first lumen carrying an optical imaging system, a second lumen for delivery of an irrigation fluid, a third lumen for delivery of a cleaning tool, a fourth lumen for applying suction, and a fifth lumen for delivery of the diverticulum remodeling agent. The catheter may be also allow passage of a diverticulum closure instrument to the diverticulum.

### BRIEF DESCRIPTION OF THE DRAWINGS

The foregoing and other features and advantages of the invention will be apparent from the following more particular description of preferred embodiments of the invention, as illustrated in the accompanying Drawings, in which:

FIG. 1 is perspective view of a walled structure of a biological organism in which a diverticulum has formed;

FIG. 2 is a perspective view of the structure of FIG. 1 showing the diverticulum following introduction of an SIS collagen gel remodeling agent therein;

FIG. 3 is a perspective view of the structure of FIG. 1 showing the diverticulum following remodeling using the SIS collagen gel remodeling agent;

FIG. 4 is a perspective view of the structure of FIG. 1 showing the diverticulum following introduction of an absorbable stent remodeling agent therein;

FIG. 5 is a perspective view of the structure of FIG. 1 showing the diverticulum following remodeling using the absorbable stent remodeling agent;

FIG. 6 is a perspective view of the structure of FIG. 1 showing the diverticulum following introduction of an absorbable spray remodeling agent therein;

FIG. 7 is a perspective view of the structure of FIG. 1 showing the diverticulum following remodeling using the absorbable spray remodeling agent;

FIG. 8 is a perspective view of the structure of FIG. 1 showing a brush cleaning implement for cleaning the diverticulum for remodeling;

FIG. 9 is a perspective view of the structure of FIG. 1 showing a staple and a stapler for closing the diverticulum following introduction of the remodeling agent;

FIG. 10 is a perspective view of the structure of FIG. 1 following closure of the diverticular neck using the staple and stapler of FIG. 7;

FIG. 11 is a perspective view of the structure of FIG. 1 showing a suture and closure clip for closing the diverticulum following introduction of the remodeling agent;

FIG. 12 is a perspective view of the structure of FIG. 1 following closure of the diverticular neck using the suture and closure clip of FIG. 11; and

FIG. 13 is a perspective view showing an endoscopic device for accessing the diverticulum shown in FIG. 1.

### DETAILED DESCRIPTION OF EXEMPLARY EMBODIMENTS

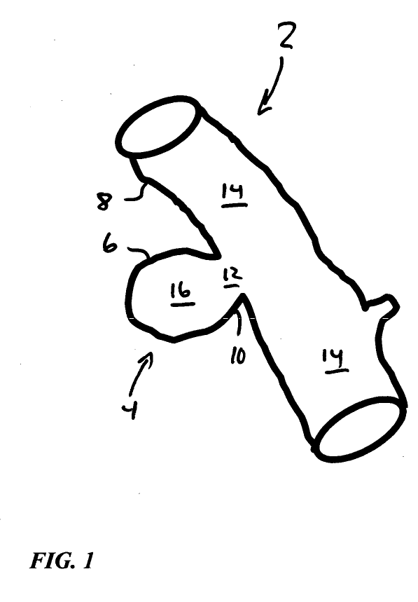

Turning now to FIG. 1, a walled structure 2 (such as a colon, small intestine, esophagus, etc.) within a biological organism is shown as having developed a diverticulum 4 caused by a region 6 of the structural wall 8 protruding into surrounding tissue (not shown) to form a sac-like structure. The diverticulum 4 includes a diverticular base 10 where the region 6 begins to balloon outwardly from the structural wall 8. The area surrounded by the diverticular base 10 represents the neck 12 of the diverticulum 4. The diverticular neck 12 leads from the hollow interior 14 of the walled structure 2 to the hollow interior of the diverticular sac 16. Unwanted material may thus enter the diverticulum 4 from the interior 12. If the structure 2 is a colon, this material creates a risk of diverticular bleeding and diverticulitis.

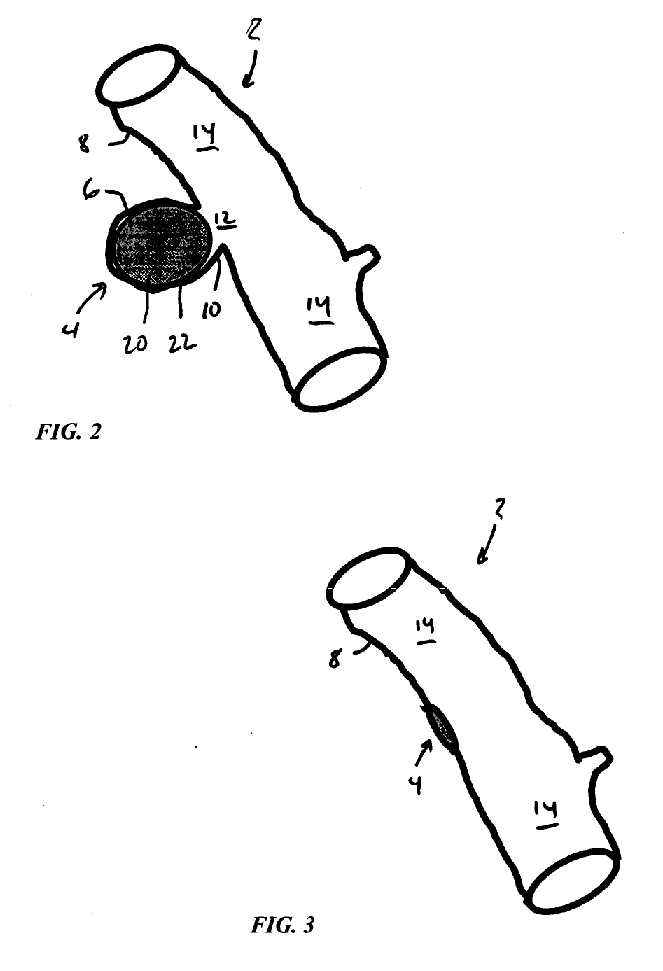

Turning now to FIG. 2, the diverticulum 4 can be remodeled by introducing an absorbable remodeling agent 20 into the diverticular sac 16 that produces shrinkage and eventual obliteration by irritating the diverticular tissue and promoting fibrosis or scar tissue growth. There are a variety of materials that may be used to provide the remodeling agent 20, including but not limited to small intestine submucosa (SIS) collagen or other absorbable fibrosis promoting materials (such as polyglycolic acid) in gel, liquid or solid form.

FIG. 2 illustrates the use of a quantity of SIS collagen gel 22 as the remodeling agent 20. SIS collagen can be harvested from animal intestines and is typically used for wound repair. Medical grade SIS collagen can be obtained in gel form from Cook Corporation of Bloomington, Ind. As shown in FIG. 2, the SIS collagen gel 22 is introduced into the diverticular sac 16 until it preferably fills or nearly fills the diverticular interior. Following optional closure of the diverticular neck 12, the SIS collagen gel 22 will promote fibrous tissue growth that contracts and effectively obliterates the diverticulum 4 over time. The diverticulum 4 following obliteration is shown in FIG. 3. To facilitate proper placement within the diverticulum 4, the SIS collagen gel 22 may have a radio-opaque dye or other visibility enhancing material incorporated therein.

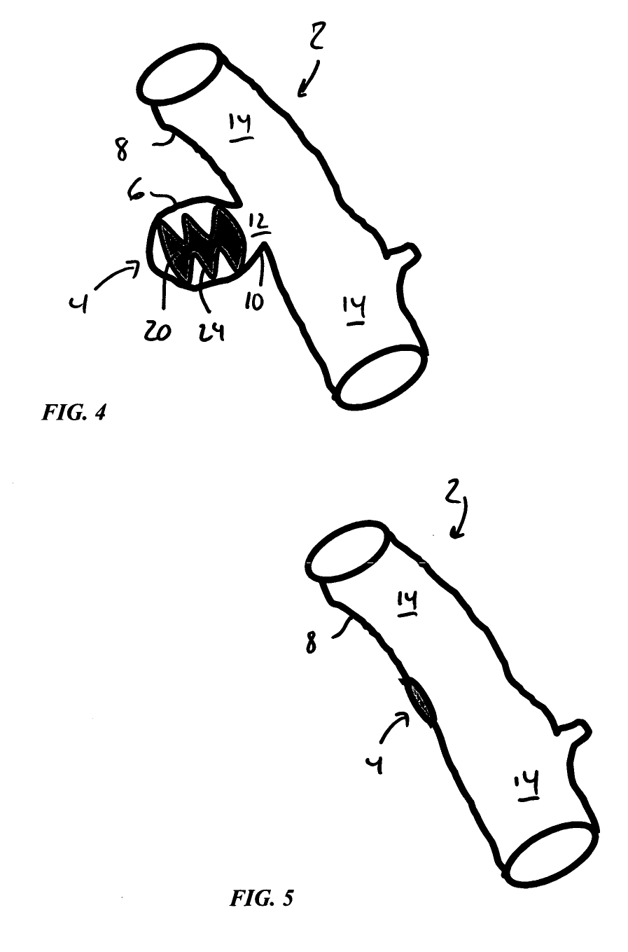

Turning now to FIG. 4, the remodeling agent 20 is implemented using a stent 24 made from an absorbable fibrosis promoting material that is solid yet flexible, such as polyglycolic acid. The stent 24 is constructed so as to be compressible in order to facilitate its insertion through the diverticular neck 12 and subsequent enlargement within the diverticular sac 16. It may be hollow or solid, open or close-ended, and may be formed into any desired shape, including but not limited to regular geometric shapes such as spheres, cylinders, or cones, or any of a variety of irregular shapes. As shown in FIG. 4, the stent 24 preferably fills or nearly fills the diverticular sac 16 following insertion therein. Following optional closure of the diverticular neck 12, the stent 24 will promote fibrous tissue growth that contracts and effectively obliterates the diverticulum 4 over time. The diverticulum 4 following obliteration is shown in FIG. 5. To facilitate proper placement within the diverticulum 4, the stent 24 may have a radio-opaque dye or other visibility enhancing material incorporated therein.

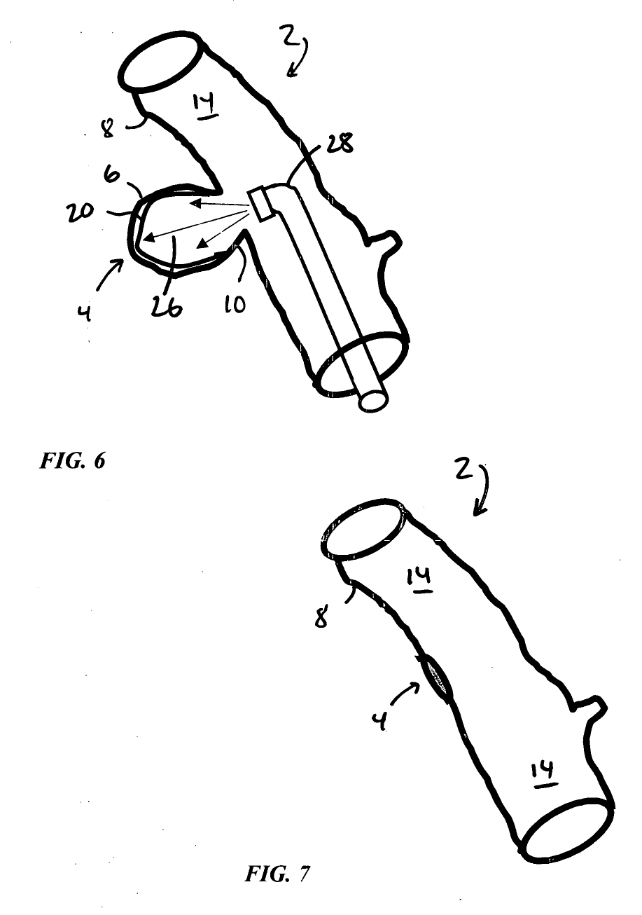

Turning now to FIG. 6, the remodeling agent 20 is implemented using a spray 26 made from a liquid absorbable fibrosis promoting material, such as SIS collagen in liquid form. The spray 26 is applied to the inside wall of the diverticular sac 16 using a spray nozzle 28. As shown in FIG. 6, the spray 26 preferably coats or nearly coats the diverticular wall following application. Following optional closure of the diverticular neck 12, the spray 26 will promote fibrous tissue growth that contracts and effectively obliterates the diverticulum 4 over time. The diverticulum 4 following obliteration is shown in FIG. 7. To facilitate proper placement within the diverticulum 4, the spray 26 may have a radio-opaque dye or other visibility enhancing material incorporated therein.

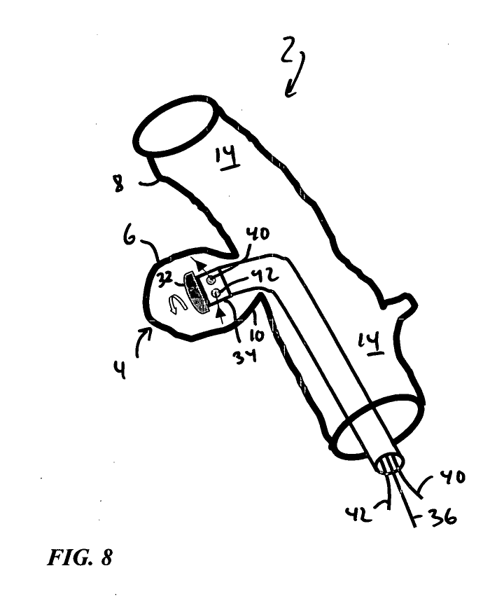

Turning now to FIG. 8, the diverticulum 4 may be optionally cleaned to remove unwanted matter therefrom prior to introduction of the remodeling agent 20. A cleaning implement configured by way of example only as a brush system 30 may be used for this purpose. The brush 30 includes rotatable brush head 32 that is mounted to a drive spindle 34 rotatably driven by a flexible drive element 36. The brush head is made of bristle material and is preferably rounded at the tip in order to prevent perforation of the diverticular wall, which may be relatively fragile. The drive element 36 is housed in a catheter 38 that extends to a location where it can be connected to a drive system (not shown) operated by medical personnel. The catheter 2 also carries a first lumen 40 adapted to deliver an irrigation fluid such as water to the diverticulum, and a second lumen 42 adapted to selectively apply suction to the diverticulum 4 to remove unwanted matter therefrom and air flow to dry the diverticulum following cleaning.

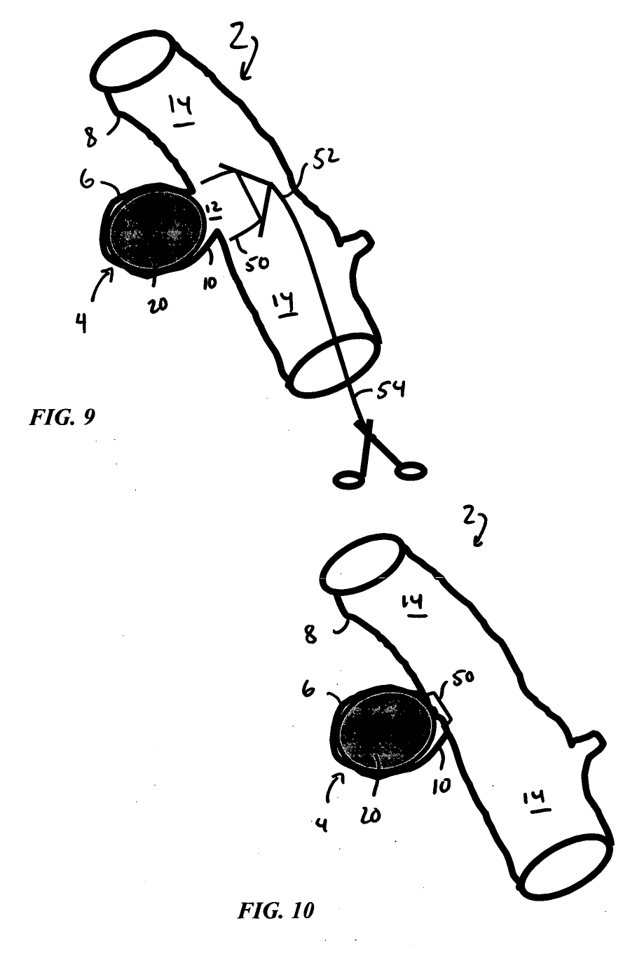

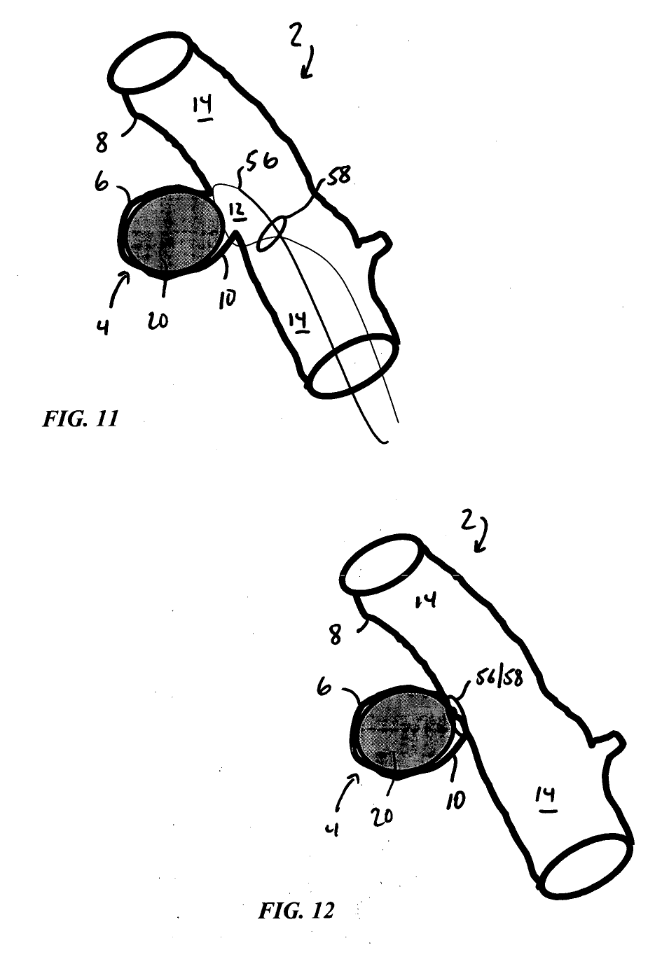

Turning now to FIGS. 9-12, the diverticular neck 12 can be closed as necessary to prevent egress of the remodeling agent 20 following its introduction into the diverticular sac 16. In FIG. 9, an absorbable staple 50 is inserted into the tissue surrounding the diverticular neck 12 and a stapler 52 having an elongated control element 54 is steered and manipulated to close the staple and thereby close the neck, as shown in FIG. 10. In FIG. 11, an absorbable suture 56 is looped through the tissue surrounding the diverticular neck 16, and an absorbable closure clip 58 is slid over the suture to clamp the suture, thereby closing the neck. Although not shown, another alternative closure means would be to introduce (as by spraying) an absorbable fibrin glue or the like into the diverticular neck. A suitable material for this purpose would be the type of fibrin glue used for the control of anastomoic leakage.

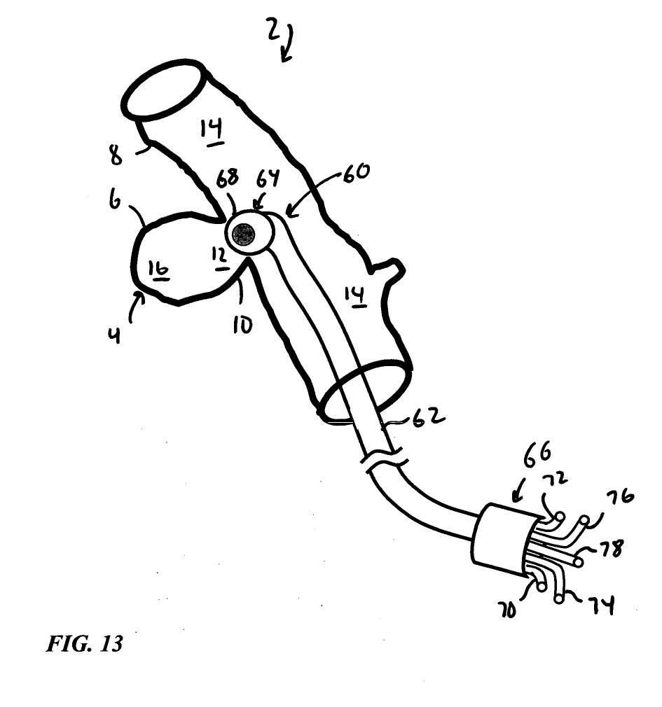

Turning now to FIG. 13, an endoscopic device 60 that may be used to access the diverticulum 4 includes a flexible catheter 62 having a steerable distal end 64 adapted to feed through the structure 2 and a proximal end 656 adapted to remain outside the patient's body. The distal end 64 is preferably bulbous (e.g., mushroom shaped), as shown at 68, in order to reduce the likelihood of puncturing the diverticular wall during insertion of the catheter 62. The catheter can be provided with one or more lumina for delivering materials and/or instruments to the diverticulum 4. In FIG. 13, the catheter 62 is shown in an exemplary configuration in which there are four lumina in which a first lumen 70 carries an optical imaging system for viewing the diverticulum 4 during remodeling, a second lumen 72 for delivery of the brush system 30 of FIG. 6, a third lumen 74 for delivery of the remodeling agent 20 (or other agents such as local anesthetic, drugs, etc.), and a fourth lumen 76 for delivery of the diverticular closure instrumentation as shown in FIGS. 9-12. A guide wire and/or steering mechanism 78 will also be typically placed in the catheter in order to guide and/or steer the catheter's distal end 64 through the structure 2.

Accordingly to an exemplary diverticular remodeling method, the diverticulum 4 is accessed non-invasively following introduction of the endoscopic device 60 through the structure 2 from outside the patient's body. If the structure 2 is the colon, the device 60 may be inserted through the rectum. If the structure 21 is the esophagus or the small intestine, the device 60 may be inserted through the mouth. Using the brush system 30, the diverticulum 4 is treated with local anesthetic and optionally cleaned to remove unwanted matter therefrom. When the diverticulum 4 is clean, the remodeling agent 20 can be introduced into the diverticular interior. The diverticular neck 12 may then be closed (as necessary) to prevent egress of the remodeling agent 20.

Accordingly, a diverticular remodeling method has been disclosed in conjunction with diverticular remodeling agents and an endoscopic device for performing the disclosed method. While various embodiments of the invention have been shown and described, it should be apparent that many variations and alternative embodiments could be implemented in accordance with the teachings herein. For example, in addition to remodeling diverticula of the colon, the small intestine and the esophagus, the technique described herein could be used to treat pancreatic cysts by way of aspiration followed by introduction of the remodeling agent 20 therein. It is understood, therefore, that the invention is not to be in any way limited except in accordance with the spirit of the appended claims and their equivalents.

What is claimed is:

1. A method for use of a composition for diverticular remodeling, comprising:
introducing a remodeling agent into a diverticulum having a diverticular sac;
said remodeling agent being adapted to promote shrinkage and obliteration of said diverticular sac.

2. A method in accordance with claim 1 further including cleaning said diverticulum to remove unwanted matter therefrom prior to introducing said remodeling agent.

3. A method in accordance with claim 2 wherein said cleaning comprises rinsing with an irrigation fluid.

4. A method in accordance with claim 2 wherein said cleaning includes brushing.

5. A method in accordance with claim 2 wherein said cleaning includes suctioning.

6. A method in accordance with claim 1 further including closing a neck of said diverticulum to retain said remodeling agent in said diverticular sac.

7. A method in accordance with claim 6 wherein said closing includes stapling a diverticular base.

8. A method in accordance with claim 6 wherein said closing includes suturing a diverticular base and clamping said suture.

9. A method in accordance with claim 1 wherein said remodeling agent comprises an absorbable fibrosis promoting gel, spray or stent.

10. A method in accordance with claim 1 wherein said remodeling agent comprises small intestine submucosa collagen.

11. A method in accordance with claim 1 wherein said remodeling agent comprises polyglycolic acid.

12. An endoscopic device for use in a method for treating diverticulosis, comprising:
a flexible catheter having a steerable distal end adapted to feed through a walled structure within a patient and a proximal end adapted to remain outside said patient's body;
a first lumen in said catheter carrying an optical imaging system;
a second lumen in said catheter for delivery of a cleaning implement;
a third lumen in said catheter for delivery of a diverticulum occluding compound;
a fourth lumen in said catheter for delivery of closure instrumentation; and
said catheter being further adapted to pass a guide wire or distal end steering mechanism;
said method including:
advancing said distal end of said endoscopic device to said diverticulum;
cleaning said diverticulum to remove unwanted matter therefrom using a cleaning tool delivered through said third lumen in combination with an irrigation fluid delivered through said second lumen and suction applied to said fourth lumen;
remodeling said diverticulum by introducing a remodeling agent into said diverticulum through said fifth lumen; and
closing said diverticulum at said colon to segregate said occluding compound from said colon.

13. A method of use of a composition for remodeling a diverticulum in a walled structure within a living organism, comprising:
accessing said diverticulum via endoscopic approach;
cleaning said diverticulum as necessary to remove unwanted matter therefrom;
introducing a remodeling agent into said diverticulum; and
closing said structure as necessary to prevent egress of said remodeling agent from said diverticulum.

14. A method in accordance with claim 13 wherein said remodeling agent comprises an absorbable fibrosis promoting material.

15. A method in accordance with claim 13 wherein said remodeling agent compound comprises small intestine submucosa collagen.

16. A method in accordance with claim 13 wherein said remodeling agent comprises an absorbable fibrosis promoting gel.

17. A device in accordance with claim 13 wherein said remodeling agent comprises an absorbable fibrosis promoting spray.

18. A method in accordance with claim 13 wherein said remodeling agent comprises an absorbable fibrosis promoting stent.

19. A method in accordance with claim 13 wherein said remodeling agent comprises a small intestine submucosa collagen gel or spray.

20. A method in accordance with claim 13 wherein said remodeling agent comprises a polyglycolic acid stent.
